(19) United States
(12) Patent Application Publication
(10) Pub. No.: US 2005/0244348 A1
(43) Pub. Date: Nov. 3, 2005

(54) Preparation to combat reddened skin

(75) Inventors: Wiebke Lindemann, Hamburg (DE); Anette Burger, Hamburg (DE); Stefan Scheede, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/079,855

(22) Filed: Mar. 14, 2005

(30) Foreign Application Priority Data
Mar. 12, 2004 (DE) 10 2004 012 135.4

(51) Int. Cl. A61K 7/42; A61K 7/021

(52) U.S. Cl. 424/59; 424/63

(57) ABSTRACT

The present invention is a cosmetic or dermatological preparation for the cosmetic treatment of reddened skin, comprising one or more green pigments, one or more white pigments and one or more UV photoprotective filters. The present invention also includes a method of treating reddened skin or skin having a tendency to redden by applying the cosmetic or dermatological preparation to the skin.

### FIELD OF THE INVENTION

The present invention relates to the use of a dermatological preparation comprising one or more green pigments, one or more white pigments and one or more UV photoprotective filters for the cosmetic treatment of reddened skin.

### BACKGROUND OF THE INVENTION

The skin is the largest human organ. It has a large number of vital functions to fulfil, for example heat regulation and the barrier function to prevent the drying out of the skin and of the entire organism, and as protective device against the penetration and the absorption of external substances. This barrier function is effected by the epidermis which, being the outermost layer, forms the actual protective sheath against the environment. Being about one tenth of the total thickness, it is also the thinnest layer of the skin.

The skin is subjected to a large number of physical, chemical and biological stresses. For various reasons, a large number of these stresses leads to a temporary or long-term reddening of the skin. These stresses include, for example, inflammations of the skin, cutaneous reactions, dermographism, soreness of the skin (for example as a result of dermatitis or contact dermatitis such as nappy dermatitis), larva migrans, erysipelas, shingles, frostbite, diphtheria, cirrhosis of the liver, gout, burns or allergic reactions.

One particular form of skin reddening here is rosacea.

Rosacea [from the Latin rosaceus >rosy<], also called copper pimples, red pimples, is a chronic disease of the facial skin which mostly arises only in middle age and in advancing years and particularly affects the nose area, also the forehead and the cheeks (butterfly shape). Dilation of the surface vessels in the skin leads to partly deep-red haemostasia, to vascular dilations (telangiectases), depending on the form also to flaking of the skin, intermittent formation of papules or pustules and (mostly only in the case of males) later to the formation of a rhinophyma. The causes are presumed to be a lability of the vascular nerves with seborrheic constitution, incretory disorders, chronic gastroenteropathy and focal infections; a genetic disposition is possible. Alcohol misuse may likewise be of significance. Treatment takes place locally with antibiotics (source: Brockhaus—Die Enzyklopädie: in 24 volumes, 20th newly revised edition, Leipzig, Mannheim: F. A. Brockhaus 1996-99).

As a rule, people affected by rosacea complain initially of localized reversible skin reddening which is caused particularly by factors such as stress and agitation, alcohol, heat, and also hot or very spicy foods and beverages. Over time, however, the condition of the skin can deteriorate and the skin is permanently reddened.

Skin reddening in general and rosacea in particular are a considerable physical and psychological worry for those affected. They are also perceived by observers in most cases as being less visually attractive, ill and unappealing.

### SUMMARY OF THE INVENTION

It was therefore the object of the present invention to develop compositions and preparations which significantly improve the visual appearance of reddened areas of skin, in particular facial skin. The aim was to develop compositions and preparations which improve the visual appearance of skin with a tendency towards reddening or skin suffering from rosacea. The improvement in the visual appearance should lie in particular in that the red areas of skin are no longer perceived as such but instead have a healthy natural appearance.

Surprisingly, the objects are achieved through the use of a cosmetic or dermatological preparation comprising
a) one or more green pigments,
b) one or more white pigments, and
c) one or more UV photoprotective filters, for the cosmetic treatment of reddened skin, in particular facial skin. Here, reddened skin is, in particular, skin suffering from rosacea.

In addition, according to the invention, it is preferably a decorative (concealing) cosmetic treatment.

It was particularly surprising that the preparation used according to the invention, which protects the skin in diverse ways against the effects of light, improves the visual appearance of skin suffering from rosacea in the long-term since phototherapy is used according to the prior art for the treatment of rosacea.

### DETAILED DESCRIPTION OF THE INVENTION

Embodiments of the invention which are advantageous according to the invention are characterized in that chromium oxide or chromium hydroxide is used as the green pigment.

Here, it is advantageous according to the invention if the green pigments are used in a total concentration of from 0.01 to 2% by weight and according to the invention it is preferred if the green pigments are used in a total concentration of from 0.1 to 1% by weight, in each case based on the total weight of the preparation.

Embodiments of the invention which are advantageous according to the invention are characterized in that titanium dioxide is used as white pigments. According to the invention, this preferably has an average particle size of at least 100 nm. For example, the titanium dioxide Cl 77891 can be used.

It is advantageous according to the invention if the white pigments are used in a total concentration of from 0.01 to 8% by weight and according to the invention it is preferred if the white pigments are used in a total concentration of from 0.5 to 5% by weight, in each case based on the total weight of the preparation.

Embodiments of the invention which are advantageous according to the invention are characterized in that one or more compounds chosen from benzylidenecamphor and derivatives thereof, e.g., 3-(4-methylbenzylidene)camphor; 4-aminobenzoic acid derivatives, preferably 2-ethylhexyl 4-(dimethylamino)benzoate and amyl 4-(dimethylamino)benzoate; esters of cinnamic acid, preferably 2-ethylhexyl 4-methoxycinnamate and isopentyl 4-methoxycinnamate; esters of salicylic acid, preferably 2-ethylhexyl salicylate, 4-isopropylbenzyl salicylate, and homomenthyl salicylate; derivatives of benzophenone, preferably 2-hydroxy-4-methoxybenzo-phenone, 2-hydroxy-4-methoxy-4′-methylbenzophenone, and 2,2′-dihydroxy-4-methoxybenzophenone; esters of benzalmalonic acid, preferably di(2-ethylhexyl) 4-methoxybenzalmalonate; 2,4,6-trianilino(p-carbo-2′-ethyl-1′-hexyloxy)-1,3,5-triazine; 2-phenylbenzimidazole-5-sulphonic acid and salts thereof, e.g., sodium, potassium or triethanolammonium salts; sulphonic acid derivatives of benzophenones, preferably 2-hydroxy-4-methoxybenzophenone-5-sulphonic acid and its salts; sulphonic acid derivatives of 3-benzylidenecamphor, such as, for example, 4-(2-oxo-3-bornylidenemethyl)benzenesulphonic acid, 2-methyl-5-(2-oxo-3-bornylidene-methyl)sulphonic acid and its salts; and derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione, are used as UV photoprotective filters.

The list of specified UV filters which can be used according to the invention is not of course intended to be limiting.

Cosmetic and dermatological preparations for the purposes of the present invention can also comprise inorganic pigments which are customarily used in cosmetics for protecting the skin against UV rays. These are oxides of titanium, zinc, iron, zirconium, silicon, manganese, aluminium, cerium and mixtures thereof, and also modifications in which the oxides are the active agents. Pigments based on titanium dioxide are particularly preferred. The amounts specified for the above combinations can be used.

According to the invention, preference is given to using the UV photoprotective filters 2-phenylbenzimidazole-5-sulphonic acid and its potassium, sodium and triethanolamine salts, 2-ethylhexyl 2-cyano-3,3-diphenylacrylate, 2-ethylhexyl 4-methoxycinnamate, 2-ethylhexyl salicylate, bis(2-ethylhexyl) 4,4-{[6-[[[(1,1-dimethyl)amino]carbonyl]phenyl]amino]-1,3,5-triazine-2,4-diyl)diimino}bisbenzoate, tris(2-ethylhexyl) 2,4,6-trianiline(p-carbo-2′-ethylhexyl-1′-oxy)-1,3,5-triazine-4,4′,4″-(1,3,5-triazine-2,4,6-triyltriimino)trisbenzoate, 2,4-bis{[4-(2-ethylhexyloxy)-2-hydroxy]-phenyl}-6-(4-methoxyphenyl)-1,3,5-triazine, 2,2′-(1,4-phenylene)bis(1H-benzimidazole-4,6-disulphonic acid, monosodium salt), 2,2′-methylenebis(6-(2H-benzotriazol-2-yl)-4-(1,1,3,3-tetramethylbutyl)phenol), 1-(4-tert-butylphenyl)-3-(4-methoxyphenyl)propane-1,3-dione, 2-hydroxy-4-methoxybenzophenone, titanium dioxide and zinc oxide.

The UV photoprotective filters 2-ethylhexyl 4-methoxycinnamate, 2-ethylhexyl salicylate and titanium dioxide are particularly preferred according to the invention.

UV photoprotective filters of titanium dioxide according to the invention differ from the titanium dioxide which is used according to the invention as white pigment by virtue of the fact that the titanium dioxide particles have an average particle size of at most 70 nm.

Preparations according to the invention can therefore advantageously comprise substances which absorb UV radiation in the UVB region, where the total amount of the filter substances is, for example, 0.1% by weight to 30% by weight, preferably 0.5 to 10% by weight, in particular 1 to 6% by weight, based on the total weight of the preparations.

The substance combination used according to the invention can advantageously be incorporated into customary cosmetic and dermatological preparations, which may be in various forms. Thus, they may, for example, be an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or a lipodispersion, a gel, a solid stick or an aerosol.

It is preferred here according to the invention if the preparation which is used according to the invention is an O/W, emulsion.

The use according to the invention is according to the invention advantageously characterized in that it comprises one or more moisturizers.

“Moisturizers” is the term used for substances or mixtures of substances which, following application or distribution on the surface of the skin, impart to cosmetic or dermatological preparations the property of reducing the moisture loss by the horny layer (also called transepidermal water loss (TEWL)) or of positively influencing hydration of the horny layer.

Advantageous moisturizers for the purposes of the present invention are, for example, glycerol; lactic acid and lactates (e.g. sodium lactate), butylene glycol, propylene glycol, biosaccaride gum-1, glycine soya, ethylhexyloxyglycerol, pyrrolidonecarboxylic acid and urea. It is also particularly advantageous to use polymeric moisturizers from the group of water-soluble, water-swellable or water-gellable polysaccharides. Particularly advantageous are, for example, hyaluronic acid, chitosan and a fucose-rich polysaccharide which is listed in the Chemical Abstracts under the registry number 178463-23-5 and is available, for example, under the name Fucogel®1000 from SOLABIA S.A. Moisturizers can also advantageously be used as anti-wrinkle active ingredients for the prophylaxis and treatment of cosmetic or dermatological changes in the skin, as arise, for example, during skin aging.

According to the invention, the total concentration of moisturizers in the preparation used according to the invention is advantageously from 1 to 20% by weight, based on the total weight of the preparation.

According to the invention, the preparation used according to the invention advantageously comprises the aqueous extract from Radix Glycyrrhizae inflatae.

The plant species Glycyrrhiza inflata belongs, like the liquorice officinal in Europe Glycyrrhiza glabra, to the genus Glycyrrhiza, which belongs to the Fabaceae plant family (pea plants). The drug Radix Glycyrrhizae inflatae, i.e., the root of the plant, is used, for example, in Far Eastern medicine.

One constituent of the aqueous extract from Radix Glycyrrhizae inflatae is licochalcone A.

It is assumed that this substance, possibly in synergy with the other constituents of the extract, has part of the effect according to the invention.

According to the invention, the preparation used according to the invention preferably comprises licochalcone A.

According to the invention, it is particularly advantageous if the preparations comprise 0.0001 to 5% by weight, in particular 0.001 to 1% by weight, very particularly 0.005 to 0.15% by weight, of licochalcone A, in each case based on the total weight of the preparation.

According to the invention, customary antioxidants can be added to preparations which comprise the active ingredient combinations according to the invention.

The antioxidants are advantageously chosen from the group consisting of amino acids (e.g., glycine, histidine, tyrosine, tryptophan) and derivatives thereof, imidazoles (e.g., urocanic acid) and derivatives thereof, peptides (e.g., D,L-carnosine, D-carnosine, L-carnosine) and derivatives thereof (e.g., anserine), carotenoids, carotenes (e.g., α-carotene, β-carotene, lycopene) and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulphoximine compounds (e.g., buthionine sulphoximines, homocysteine sulphoximine, buthionine sulphones, penta-, hexa-, heptathionine sulphoximine) in very low tolerated doses (e.g., pmol to μmol/kg), and also (metal) chelating agents (e.g., α-hydroxy fatty acids, palmitic acid, phytic acid, and lactoferrin), α-hydroxy acids (e.g., citric acid, lactic acid, and malic acid), humic acid, bile acid, bile extracts, bilirubin, biliverdin, EDTA, EGTA and derivatives thereof, unsaturated fatty acids and derivatives thereof (e.g., γ-linolenic acid, linoleic acid, and oleic acid), folic acid and derivatives thereof, alaninediacetic acid, flavonoids, niacinamide, panthenol, polyphenols, catechins, vitamin C and derivatives (e.g., ascorbyl palmitate, Mg ascorbyl phosphate, and ascorbyl acetate), tocopherols and derivatives (e.g., vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g., ZnO, ZnSO₄), selenium and derivatives thereof (e.g., selenomethionine), stilbenes and derivatives thereof (e.g., stilbene oxide, trans-stilbene oxide) and the derivatives (salts, esters, ethers, sugars, nucleotides, nucleosides, peptides and lipids) suitable according to the invention of these specified active ingredients.

The amount of antioxidants (one or more compounds) in the preparations is preferably 0.001 to 30% by weight, particularly preferably 0.05-20% by weight, in particular 1-10% by weight, based on the total weight of the preparation.

The preparations used according to the invention are used in the customary manner, namely by applying the preparation according to the invention to the areas of affected skin.

According to the invention, it may be advantageous to add further agents effective in combating skin reddening or rosacea to the preparations according to the invention, for example chosen from the group of anti-inflammatory substances, such as, for example, allantoin, tannin, antihistamines, antiphlogistics, glucocorticoids (e.g., hydrocortisone), polidocanol, and plant active ingredients such as azulene, bisabolol, glycyrrhizin, hamamelis and plant extracts such as camomile. According to the invention, preference is given here to the compounds bisabolol.

One or more of these anti-inflammatory substances may, according to the invention, advantageously be present in the preparation in a total concentration of from 0.01 to 5% by weight, preferably in a concentration of from 0.1 to 1% by weight and very particularly preferably in a concentration of from 0.2 to 0.75% by weight, in each case based on the total weight of the preparation.

The cosmetic and dermatological preparations used according to the invention can comprise cosmetic active ingredients, auxiliaries or additives as are customarily used in such preparations, e.g., consistency regulators, fillers, antioxidants, preservatives, bactericides, perfumes, substances for preventing foaming, dyes, pigments which have a colouring effect, thickeners, vitamins, proteins, surface-active substances, emulsifiers, softening, moisturizing or humectant substances, fats, oils, waxes or other customary constituents of a cosmetic or dermatological formulation, such as alcohols, polyols, polymers, foam stabilizers, electrolytes, organic solvents or silicone derivatives.

The preparations used according to the invention can also advantageously comprise:
water or aqueous solutions;
oils, such as triglycerides of capric or of caprylic acid, but preferably castor oil;
fats, waxes and other natural and synthetic fatty substances, preferably esters of fatty acids with alcohols of low carbon number, e.g., with isopropanol, propylene glycol or glycerol, or esters of fatty alcohols with alkanoic acids of low carbon number or with fatty acids; and
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethylether and analogous products.

The oil phase of the emulsions, oleogels, hydrodispersions or lipodispersions for the purposes of the present invention is advantageously chosen from the group of esters of saturated or unsaturated, branched or unbranched alkanecarboxylic acids with a chain length of from 3 to 30 carbon atoms and saturated or unsaturated, branched or unbranched alcohols with a chain length of from 3 to 30 carbon atoms, from the group of esters of aromatic carboxylic acids and saturated or unsaturated, branched or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g., jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated or unsaturated, branched or unbranched alkanecarboxylic acids with a chain length of from 8 to 24, in particular 12-18, carbon atoms. The fatty acid triglycerides can, for example, advantageously be chosen from the group of synthetic, semisynthetic and natural oils, e.g., olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. It may in some instances also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic-capric triglyceride, dicaprylyl ether.

Mixtures of C₁₂₋₁₅-alkyl benzoate and 2-ethylhexyl isostearate, mixtures of C₁₂₋₁₅ alkyl benzoate and isotridecyl isononanoate, and mixtures of C₁₂₋₁₅ alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of cyclic or linear silicone oils or consist entirely of such oils, although it is preferred to use an additional content of other oil phase components apart from the silicone oil or the silicone oils.

Advantageously, cyclomethicone(octamethylcyclotetrasiloxane) is used as silicone oil to be used according to the invention. However, other silicone oils are also to be used advantageously for the purposes of the present invention, for example hexamethylcyclotrisiloxane, polydimethylsiloxane, and poly(methylphenylsiloxane).

Also particularly advantageous are mixtures of cyclomethicone and isotridecyl isononanoate, and of cyclomethicone and 2-ethylhexyl isostearate.

Customary basic substances which are suitable for use as cosmetic sticks for the purposes of the present invention are liquid oils (e.g., paraffin oils, castor oil, and isopropyl myristate), semisolid constituents (e.g., vaseline and lanolin), solid constituents (e.g., beeswax, ceresine and microcrystalline waxes, and ozokerite), and high-melting waxes (e.g., carnauba wax and candelilla wax).

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl and monobutyl ether, propylene glycol monomethyl, monoethyl or monobutyl ether, diethylene glycol monomethyl or monoethyl ether and analogous products, and also alcohols of low carbon number, e.g., ethanol, isopropanol, 1,2-propanediol, glycerol, and in particular one or more thickeners, which can advantageously be chosen from the group consisting of silicon dioxide, aluminium silicates, polysaccharides and derivatives thereof, e.g., hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

Gels used according to the invention usually comprise alcohols of low carbon number, e.g., ethanol, isopropanol, 1,2-propanediol, glycerol and water or an above-mentioned oil in the presence of a thickener which, in the case of oily-alcoholic gels, is preferably silicon dioxide or an aluminium silicate, and in the case of aqueous-alcoholic or alcoholic gels, is preferably a polyacrylate.

Cosmetic preparations for the purposes of the present invention can also be in the form of gels which, besides an effective content of the active ingredient according to the invention and solvents customarily used therefor, preferably water, also comprise organic thickeners, e.g., gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g., aluminium silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

Last but not least, the preparations according to the invention comprising the extract or active ingredient according to the invention can advantageously be used according to the invention for impregnating textiles (e.g., wipes, pads) or plasters (e.g., as active ingredient plasters).

It is advantageous to buffer the compositions according to the invention. A pH range from 3.5-7.5 is advantageous. It is particularly favourable to choose the pH in a range from 4.0-6.5.

The particle size distribution of the titanium dioxides can according to the invention be advantageously determined by the following method:

Titanium dioxide is dispersed in water at room temperature and treated with ultrasound for 3 min in order to destroy agglomerates. The particle size distribution is then determined by means of static laser light diffraction and the particle size distribution is calculated in accordance with the Fraunhofer diffraction model.

The examples below are intended to illustrate the present invention. Unless stated otherwise, the data always refer to % by weight.

### EXAMPLES

### Examples 1-10

### OW Creams

| | Example number | | | | |
|---|---|---|---|---|---|
| | 1 | 2 | 3 | 4 | 5 |
| Glyceryl stearate citrate | 2 | | | 2 | |
| Glyceryl stearate, self-emulsifying | | 5 | 3 | | 2 |
| PEG-40 stearate | | | 1 | | 1 |
| Myristyl myristate | 1 | | | | 1 |
| Stearyl alcohol | 2 | 1 | | | |
| Cetearyl alcohol | | | | 4 | 2 |
| Cetyl alcohol | 1 | | 3 | | |
| Hydrogenated coconut fatty glycerides | 2 | | | | |
| (hydrogenated cocoglycerides) | | | | | |
| Butyrospermium Parkii | | 2 | | 4 | |
| C12-15 alkyl benzoate | | 3 | 2 | | 3 |
| Squalanes | 1 | | | 1 | |
| Caprylic/capric triglyceride | | 1 | 1 | 2 | 2 |
| Ethylhexyl coconut fatty acid ester | 3 | | | | 1 |
| Octyldodecanol | | | 1 | | |
| Octamethyltetrasiloxane(cyclomethicone) | 4 | | | | |
| Dimethylpolysiloxane(dimethicone) | | | 1 | 3 | |
| Dicaprylyl ether | 1 | 4 | 2 | | |
| Dicaprylyl carbonate | | | | 3 | |
| Titanium dioxide (<40 nm) | | 2 | 1 | 2 | |
| Zinc oxide | | | 2 | | |
| Octyltriazone | | 3 | | 1 | |
| Ethylhexyl methoxycinnamate | 5 | 7.5 | 4 | | 6 |
| Ethylhexyl cyanodiphenylacrylate | | | | 5 | |
| (octocrylene) | | | | | |
| Bisethylhexyloxyphenol | | 2 | | 3 | |
| methoxyphenyltriazine | | | | | |
| Bisimidazylate | | | | | 2 |
| Ethylhexyl salicylate | 3 | | | | 1 |
| Tetramethylbutylphenol | | 2 | | | |
| Butylmethoxydibenzoylmethane | | | 2 | | |
| Benzophenone-3 | | | 2 | | |
| Phenylbenzimidazolesulphonic acid | 1 | | | | 3 |
| Diethylhexylbutylamidotriazone | | | 0.5 | | 1 |
| Glycolic acid | | | | 1 | |
| Bisabolol | 0.5 | | | 0.3 | |
| Tocopheryl acetate | | | 1 | | |
| Glycyrrhiza Inflata Root Extract | 0.1 | 0.02 | 0.01 | | 0.07 |
| Citric acid, sodium salt | | 0.3 | | | |
| Sodium ascorbyl phosphate | 0.1 | | | | |
| Trisodium EDTA | | 0.2 | | 0.1 | |
| Iminodisuccinate, sodium salt | 0.2 | | | | |
| Phenoxyethanol | 0.3 | | 0.3 | 0.2 | 0.2 |
| p-Hydroxybenzoic alkyl ester | 0.6 | 0.3 | | 0.3 | 0.3 |
| (paraben) | | | | | |
| Hexamidine diisethionate | | 0.04 | | | |
| Diazolidinylurea | 0.25 | | 0.1 | | 0.1 |
| 1,3-Dimethylol-5,5-dimethyl-hydantoin | | 0.2 | | | |
| (DMDM hydantoin) | | | | | |
| Iodopropynyl butylcarbamate | | 0.1 | | | |
| Ethanol denatured | | 2 | | | |
| Xanthan gum | 0.1 | | | 0.2 | |
| Polyacrylic acid (Carbomer) | | | 0.1 | | 0.1 |
| Polyacrylamide | | 0.2 | | | |
| Glycerol | 10 | 6 | 6 | 7.5 | 8 |
| Butylene glycol | 2 | 1 | 0.5 | | 0.5 |
| Chromium oxide (Cl 77288) | 0.01 | 0.2 | 0.05 | 0.3 | 0.7 |
| Chromium oxide, hydrous (Cl 77289) | 0.1 | 0.2 | 0.05 | 0.5 | 0.5 |
| Titanium dioxide (Cl 77891) | 0.5 | 4 | 5 | 2 | 1 |
| Ultramarine (Cl 77007) | 0.1 | 0.1 | | | 0.2 |
| Fillers/additives (distarch phosphate, | 0.1 | 1 | 0.2 | 0.5 | 0.05 |
| tapioca starch, SiO₂, BHT, talc, | | | | | |
| aluminium stearate) | | | | | |
| Neutralizing agent | q.s. | q.s. | q.s. | q.s. | q.s. |
| Perfume | q.s. | q.s. | q.s. | q.s. | q.s. |
| Water | ad | ad | ad | ad | ad |
| | 100 | 100 | 100 | 100 | 100 |

| | Example number | | | | |
|---|---|---|---|---|---|
| | 6 | 7 | 8 | 9 | 10 |
| Glyceryl stearate, self-emulsifying | 2.5 | | | | |
| PEG-40 stearate | 1 | | | | |
| Polyglyceryl-3-methylglucose | | 3 | | | |
| distearate | | | | | |
| Sorbitan stearate | | 1 | | | |
| Polyethylene glycol(21) stearyl | | | 2 | | |
| ether (steareth-21) | | | | | |
| Polyethylene glycol(2) stearyl | | | 1 | | |
| ether (steareth-2) | | | | | |
| Cetearyl glucoside | | | | 2 | |
| Stearic acid | | | | | 2.5 |
| Myristyl myristate | | | | 1 | |
| Behenyl alcohol | | 1 | | | 2 |
| Stearyl alcohol | | | | 3 | |
| Cetearyl alcohol | 3 | | 2 | | 1 |
| Cetyl alcohol | | 1 | | | |
| C12-15 alkyl benzoate | 4 | 2 | 5 | 2 | |
| Butylene glycol dicaprylate/dicaprate | | | | | 2 |
| Ethylhexyl coconut fatty acid ester | | | | | 2 |
| Octyldodecanol | | 4 | 1 | | 1 |
| Lactic acid | | | 1 | | |
| Tocopheryl acetate | | | | 2 | |
| Salicylic acid | | | | 0.5 | |
| Octamethyltetrasiloxane(cyclomethicone) | 4 | 3 | 2 | | 2 |
| Dimethylpolysiloxane(dimethicone) | | | | | 1 |
| Dicaprylyl ether | | | 2 | | |
| Dicaprylyl carbonate | | 2 | | 3 | 4 |
| Titanium dioxide (<40 nm) | 1 | | 3 | 1 | 2 |
| Zinc oxide | | 2 | | | |
| Octyltriazone | 2 | | | | |
| Benzophenone-3 | 4 | | | 5 | |
| Ethylhexyl methoxycinnamate | 5 | 7.5 | 4 | | 6 |
| Ethylhexyl cyanodiphenylacrylate | | | 2 | | |
| (octocrylene) | | | | | |
| Bisethylhexyloxyphenol | | | 1 | | |
| methoxyphenyltriazine | | | | | |
| Bisimidazylate | | | | | 2 |
| Ethylhexyl salicylate | 3 | | | 3 | |
| Tetramethylbutylphenol | | | | | 2 |
| Butylmethoxydibenzoylmethane | | 2 | | | |
| Phenylbenzimidazolesulphonic acid | 1 | | | | |
| Diethylhexylbutylamidotriazone | | | 0.5 | | |
| Glycyrrhiza Inflata Root Extract | 0.05 | 1 | | 0.2 | |
| Dexpanthenol | | 1 | | | 5 |
| Bisabolol | 1 | | | 0.2 | |
| Allantoin | | | 0.5 | | 1 |
| Lactoferrin | | | | | 0.05 |
| Trisodium EDTA | | | 0.2 | 0.1 | |
| Iminodisuccinate | 0.2 | | | | 0.1 |
| Phenoxyethanol | 0.5 | 0.4 | 0.5 | | 0.3 |
| p-Hydroxybenzoic alkyl ester | 0.1 | | | 0.4 | 0.6 |
| (paraben) | | | | | |
| Hexamidine diisethionate | | | 0.1 | | |
| Diazolidinylurea | 0.2 | 0.2 | | 0.1 | |
| Iodopropynyl butylcarbamate | | | 0.25 | | |
| Ethanol denatured | | 8 | | | 3 |
| 2-Ethylhexyl glycerol ether | | | | 0.4 | |
| (octoxyglycerol) | | | | | |
| Xanthan gum | | 0.1 | | | |
| Polyacrylic acid (Carbomer) | 0.2 | | 0.1 | | 0.1 |
| Polyacrylamide | | 0.2 | | | |
| Glycerol | 10 | 5 | 6 | 3 | 7 |
| Butylene glycol | 0.5 | 1 | | 4 | |
| Chromium oxide (Cl 77288) | 0.2 | 0.5 | | 0.5 | 0.4 |
| Chromium oxide, hydrous (Cl 77289) | 0.1 | | 0.4 | 0.5 | 0.1 |
| Titanium dioxide (Cl 77891) | 0.1 | 1 | 4 | 3 | 2 |
| Ultramarine (Cl 77007) | | 0.1 | | 0.05 | |
| Additives (distarch phosphate, SiO₂, | 0.03 | 0.1 | 0.05 | 3 | 1 |
| talc, BHT aluminium stearate) | | | | | |
| Neutralizing agent | q.s. | q.s. | q.s. | q.s. | q.s. |
| Perfume | q.s. | q.s. | q.s. | q.s. | q.s. |
| Water | ad | ad | ad | ad | ad |
| | 100 | 100 | 100 | 100 | 100 |

### Example 11

### Hydrodispersion/Gel Cream

| | |
|---|---|
| Cetyl alcohol | 2 |
| Octyldodecanol | 4 |
| Octamethyltetrasiloxane (cyclomethicone) | 5 |
| Dimethylpolysiloxane (dimethicone) | 1 |
| Titanium dioxide (Cl 77891) | 1 |
| Butylmethoxydibenzoylmethane | 2 |
| Octyl methoxycinnamate | 5 |
| Octyl salicylate | 3 |
| Biotin | 0.03 |
| Iminodisuccinate | 0.2 |
| Phenoxyethanol | 0.3 |
| p-Hydroxybenzoic alkyl ester (paraben) | 0.4 |
| Xanthan gum | 0.2 |
| Glycerol | 5 |
| Chromium oxide (Cl 77288) | 0.5 |
| Chromium oxide, hydrous (77007) | 0.2 |
| Glycyrrhiza Inflata Root Extract | 0.025 |
| Perfume | q.s. |
| Neutralizing agent | q.s. |
| Water | ad 100 |

1. A cosmetic or dermatological preparation for the treatment of reddened skin or skin having a tendency towards reddening, comprising
a) one or more green pigments;
b) one or more white pigments; and
c) one or more UV photoprotective filters.

2. The preparation according to claim 1, wherein the one or more green pigments include one or more of chromium oxide and chromium hydroxide.

3. The preparation according to claim 1, wherein the one or more white pigments include titanium dioxide having an average particle size of at least 100 nm.

4. The preparation according to claim 1, wherein the one or more UV photoprotective filters are selected from the group consisting of 2-phenylbenzimidazole-5-sulphonic acid and its potassium, sodium and triethanolamine salts; 2-ethylhexyl 2-cyano-3,3-diphenylacrylate; 2-ethyl hexyl 4-methoxycinnamate; 2-ethyl hexyl salicylate; bis(2-ethylhexyl) 4,4-{[6-[[[(1,1-dimethyl)amino]carbonyl]phenyl]amino]-1,3,5-triazine-2,4-diyl)diimino}bisbenzoate; tris(2-ethylhexyl) 2,4,6-trianiline(p-carbo-2′-ethylhexyl-1′-oxy)-1,3,5-triazine-4,4′,4″-(1,3,5-triazine-2,4,6-triyltriimino)trisbenzoate; 2,4-bis{[4-(2-ethylhexyloxy)-2-hydroxy]phenyl }-6-(4-methoxyphenyl)-1,3,5-triazine; 2,2′-(1,4-phenylene)bis(1H-benzimidazole-4,6-disulphonic acid, monosodium salt); 2,2′-methylenebis(6-(2H-benzotriazol-2-yl)-4-(1,1,3,3-tetramethylbutyl)phenol); 1-(4-tert-butylphenyl)-3-(4-methoxyphenyl)propane-1,3-dione; 2-hydroxy-4-methoxybenzo-phenone, titanium dioxide and zinc oxide.

5. The preparation according to claim 1, wherein the one or more UV photoprotective filters include one or more UV photoprotective filters selected from the group consisting of 2-ethylhexyl 4-methoxycinnamate, 2-ethylhexyl salicylate and titanium dioxide.

6. The preparation according to claim 1, in the form of an oil-in-water emulsion.

7. The preparation according to claim 1, wherein the one or more green pigments are present in a total concentration of from 0.01 to 2% by weight, based on the total weight of the preparation.

8. The preparation according to claim 1, wherein the one or more white pigments are present in a total concentration of from 0.01 to 8% by weight, based on the total weight of the preparation.

9. The preparation according to claim 1, wherein the one or more UV photoprotective filters are present in a total concentration of from 0.1 to 30% by weight, based on the total weight of the preparation.

10. The preparation according to claim 1, further comprising one or more moisturizers.

11. The preparation according to claim 1, further comprising an aqueous extract from Radix Glycyrrhizae inflatae.

12. The preparation according to claim 1, further comprising licochalcone A.

13. A method for the treatment of reddened skin or skin having a tendency towards reddening, comprising applying to the skin a cosmetic or dermatological preparation comprising a) one or more green pigments, b) one or more white pigments, and c) one or more UV photoprotective filters.

14. The method according to claim 13, for the treatment of skin suffering from rosacea.

15. The method according to claim 13, wherein the applying step comprises applying the preparation to facial skin.

16. The method according to claim 13, wherein the one or more green pigments include one or more of chromium oxide and chromium hydroxide.

17. The method according to claim 13, wherein the one or more white pigments include titanium dioxide having an average particle size of at least 100 nm.

18. The method according to claim 13, wherein the one or more UV photoprotective filters include one or more UV photoprotective filters selected from the group consisting of 2-ethylhexyl 4-methoxycinnamate, 2-ethylhexyl salicylate and titanium dioxide.

19. The method according to claim 13, wherein the one or more green pigments are present in a total concentration of from 0.01 to 2% by weight, the one or more white pigments are present in a total concentration of from 0.01 to 8% by weight, and the one or more UV photoprotective filters are present in a total concentration of from 0.1 to 30% by weight, in each case based on the total weight of the preparation.

20. The method according to claim 13, wherein the preparation further comprises one or more moisturizers.

21. The method according to claim 13, wherein the preparation further comprises an aqueous extract from Radix Glycyrrhizae inflatae.

22. The method according to claim 13, wherein the preparation further comprises licochalcone A.
